(19) United States
(12) Patent Application Publication
(10) Pub. No.: US 2025/0213197 A1
(43) Pub. Date: Jul. 3, 2025

(54) FORWARD BACKWARD FOURIER TRANSFORM (FBFT) MODEL FOR BRAIN DISEASE DETECTION

(71) Applicant: Hamad Bin Khalifa University (HBKU), Doha (QA)

(72) Inventors: Nisreen S. Amer, Doha (QA); Samir Brahim Belhaouari, Doha (QA)

(21) Appl. No.: 18/980,500

(22) Filed: Dec. 13, 2024

Related U.S. Application Data
(60) Provisional application No. 63/612,190, filed on Dec. 19, 2023.

(51) Int. Cl. A61B 5/00; G16H 50/20

(52) U.S. Cl. CPC A61B 5/7257; A61B 5/0042; A61B 5/726; G16H 50/20

(57) ABSTRACT

The present disclosure provides for a forward backward Fourier transform (FBFT) model for brain disease detection. According to one aspect of the present disclosure a forward backward Fourier transform (FBFT) model for brain disease detection. According to a second aspect of the present disclosure a method of using a forward backward Fourier transform (FBFT) model for brain disease detection.

### CROSS-REFERENCE TO RELATED APPLICATIONS

The present application claims the benefit of U.S. Provisional Application No. 63/612,190 filed Dec. 19, 2023, which is incorporated herein by reference in its entirety.

### BACKGROUND

The brain is a complex organ responsible for a wide range of cognitive, emotional, and physical functions. However, numerous diseases can affect the brain and impair its normal functioning. These diseases include traumatic brain injury, infections, degenerative conditions like Alzheimer's disease and other forms of dementia, and Parkinson's disease. Brain disorders are conditions that significantly impact brain function and can arise from various causes such as genetic factors, physical trauma, infections, or environmental influences.

The impact of brain diseases on individuals and society is significant. For example, Alzheimer's and other dementias resulted in approximately 258,600 deaths in the US in 2017 alone. The number of people affected by Alzheimer's disease and related dementias is projected to increase to 9.3 million by 2060. Similarly, epilepsy affects about 50 million people worldwide, and about 80% of people with epilepsy in developing countries do not receive the treatment they need.

Early detection and precise identification of brain conditions are crucial for saving lives and reducing the symptoms and disabilities associated with these disorders. EEG signals, which represent brain activity, play a vital role in biomedical health-care. They are particularly important for the identification of epilepsy, Alzheimer's, mental stress, autism, ADHD, and other brain and neurological disorders. Electroencephalogram (EEG) analysis is a key part of the medical diagnosis of neurological disorders. However, for medical practitioners, it is usually very difficult to interpret complex EEG signals and diagnose brain disorders directly from them. Furthermore, EEG signals are of very low amplitude and often contaminated by noise sources. Various techniques, such as amplification and denoising, are applied to enhance and clean the signals. Further filtering and processing methods are then employed based on the specific application. Researchers are continuously studying EEG signals to improve pre-processing methods and feature extraction techniques, enabling the extraction of reliable features that consider the complexity of brain dynamics.

Previous studies have attempted to extract a variety of linear and nonlinear EEG features from the time, frequency, and time-frequency domains. These features can be used to characterize the electrical activity of the brain in order to better understand its underlying mechanisms. Recent development in the frequency domain analysis aimed at improving detection accuracy by reducing the dimensionality of EEG data using a non-linear method and channel selection. The study divided EEG recordings from 23 patients in the CHB-MIT database into 10-second segments, and calculated the Spectral Power Density for 5 basic EEG rhythms for each of the 23 channels. Feature selection was then performed using the Random Forests algorithm to determine the best channels, reducing the spatial information to 3 channels. The t-distributed Stochastic Neighbor Embedding (tSNE) method was applied to further limit the selected features, resulting in a 2-dimensional representation of the data. Finally, a kNN classifier was used to solve the seizure-non seizure problem and achieved a sensitivity of 80.87%. Similarly, the literature presented a method of reducing the dimensionality of EEG data using a Convolutional Neural Network (CNN). The research utilized EEG recordings from 23 patients in the CHB-MIT database and divided them into 1-second segments. FFT and WPD were then used to calculate the time, frequency, and TF characteristics. These characteristics were fed into a CNN to identify the best features for classification. The classification model was based on a TSK fuzzy system and achieved accuracy (ACC), sensitivity (SENS), and specificity (SPEC) rates of 98.30%, 96.70%, and 99.10% respectively.

The literature introduced a method for seizure detection. The process involves dividing EEG recordings into 10-second segments with a 5-second overlap and using the Fast Fourier Transform (FFT) to determine the power of 5 EEG rhythms. A threshold function is then applied to differentiate between pathological and non-pathological sections. To study the progression from pre-ictal to ictal period, a network of distances was created based on the Euclidean distance between the coefficients of the Fourier Transform in the high band of rhythm c. This network represents the synchronization of brain activity and spread of a seizure. A network of correlations was also established between the channels. The method was tested on EEG recordings from 18 patients and achieved a sensitivity rate of 83%.

A novel approach was introduced by the literature for the ictalinterictal classification problem in epilepsy. The method involves using the Discrete Fourier Transform (DFT) to extract band energies, which were then input into an Attention Network AttVGGNet. The approach achieved high accuracy rates, with ACC=95.6%, SENS=94.7%, SPEC=94.1%, Recall=89.3%, and Precision=78.1%.

The literature developed a method for improving the accuracy of epilepsy diagnosis using EEG recordings. The approach combined the Discrete Fourier Transform (DFT) with effective brain connectivity measures, which were then fed into an Autoencoder Neural Network. The results showed high accuracy, with ACC=97.91%, SENS=97.65%, and SPEC=98.06%.

Similarly, the literature used a Convolutional Neural Network (CNN) to classify EEG recordings in the ictalinterictal problem. The recordings from 23 patients in the CHB-MIT database were separated into 1-second segments and FFT and WPD were used to extract time, frequency, and TF characteristics. These characteristics were then fed into the CNN for dimension reduction and classification using a TSK fuzzy system. The methodology produced high accuracy results with ACC=98.30%, SENS=96.70%, and SPEC=99.10%.

Another development was the exploration of the time-frequency feature extraction, for example, Short-Time Fourier Transform has been used in a few EEG-based epilepsy detection studies. The literature introduced a method that uses spectral features to train a CNN. They utilized the Short-Time Fourier Transform (STFT) to calculate the spectrum amplitude from different frequency subbands and then employed a two-layer CNN to choose and merge the most informative features. The selected features were then used to train an Extreme Learning Machine (ELM) neural network. The proposed method was evaluated using the CHB-MIT database for three different classification problems: ic-tal/interictal, ictal/interictal/preictal, and ictal/interictal/preictal status 1/preictal status 2/preictal status 3, resulting in high classification rates of 99.33%, 98.62%, and 87.95%, respectively.

Similarly, the literature used spectrogram and scalogram images obtained from the Short Time Fourier Trans-form (STFT) to train a Convolutional Neural Network (CNN), which achieved an accuracy of 97% in the ictal-preictal-interictal classification problem. Other researchers also proposed using CNNs to classify EEG recordings obtained from STFT. The literature achieved an accuracy of 91.71% in the ictalinterictal classification task, with sensitivity and specificity scores of 91.09% and 94.73%, respectively, using the leave-one-subject-out cross-validation method. Literature has achieved an accuracy of 97.75% in the ictalinterictal classification problem, with recall and precision scores of 98.44% and 97.47%, respectively.

On the other hand, despite its increased complexity, wavelet analysis is widely used due to its superior performance compared to other methods. The literature used the Scattering Transform method to detect abnormalities in EEG recordings and identify seizure regions. The method is based on the combination of the Wavelet Transform and CNN. The literature applied the technique to 24 patients from the CHB-MIT database, finding the best window length to be 2 seconds (512 points) with 50% coverage. The unsupervised classification method achieved an accuracy of 91.40% in correctly classifying 180 out of 197 seizures.

Similarly, the literature proposed a seizure detection method using DWT, Shannon entropy, and kNN. DWT is applied to EEG recordings to calculate Shannon entropy and standard deviation for each frequency and for the whole spectrum. The resulting characteristics are fed into a kNN classifier. The method was tested on 10 CHB-MIT patients and achieved a SENS of 94.50%. Literature utilized the Discrete Wavelet Transform and a Nonlinear Vector Decomposed Neural Network to classify ictal and interictal EEG signals. The methodology achieved high accuracy results, with ACC=95.6%, SENS=94.7%, SPEC=94.1%, Recall=89.3%, and Precision=78.1%. The literature presented an adaptive approach using the Pattern Wavelet Transform and a Fuzzy classifier for seizure detection, achieving ACC of 96.02% and SPEC of 94.5% in the ictalinterictal classification problem.

In a recent study, the literature used a combination of DWT and LDA classifier to address the ictal-interictal problem, resulting in an ACC of 99.6% and a SENS of 99.8%. In the literature, a novel approach for epileptic seizure detection using EEG data was proposed. The approach involved using a spatiotemporal graph attention network (STGAT) based on synchronization. To extract the spatial and functional connectivity information between EEG channels, the phase locking values (PLVs) were first calculated. This allowed the multichannel EEG signals to be modeled as graph signals. The STGAT model was then used to dynamically learn the temporal correlation properties of EEG sequences and to uncover the spatial topological structure information of multiple channels. The results of experiments on two benchmark datasets showed that the STGAT model was successful in capturing the spatiotemporal correlations present in EEG data. On the CHB-MIT dataset, the model achieved a high level of accuracy with 98.74% accuracy, 99.21% specificity, and 98.87% sensitivity.

The literature proposed multiscale short-time Fourier transform as a novel feature extraction method. The 3D convolution neural network architecture was designed to accurately capture predictive probabilities of samples, and a rectified weighting strategy was proposed to enhance predictive probabilities. An accumulative decision-making rule was also introduced to achieve short detection latency. The results showed that the proposed algorithm achieved 94 out of 99 seizures detected during the crossing period, with an average 14.84% rectified predictive ictal probability error and a 2.3 s detection latency.

Several studies have demonstrated encouraging outcomes in the identification of neurological disorders like Alzheimer's disease. Although there no specific cure for AD, the timely identification of the condition may help in enhancing the quality of life for those affected. The literature implemented a methodology that leverages techniques for extracting distinctive attributes and categorizing EEG. They differentiate between patients afflicted AD, those experiencing mild AD, and individuals in a healthy group. A total of 109 samples spanning AD, MCI, and HC categories are converted to scalograms using both Fourier and Wavelet Transforms. Through the utilization of Wavelet-based feature extraction, they attained classification accuracies of 83% for AD and normal cases, 92% for health and mild AD cases, and 79% for Mild and AD classification scenarios.

In literature, authors employed six computational techniques for analyzing time-series data i.e. EEG of 160 subjects with AD and 24 with HC. Findings derived from both the original and wavelet-filtered EEG signals to sub-bands indicate that some of the validated methods, such as wavelet-coherence and quantile graphs, exhibit a robust capacity to differentiate between AD patients and healthy elderly participants with high accuracy. The literature proposed graph theoretical approaches to analyze brain functional or cortical connectivity from EEG signals. Brain networks were modeled as graphs based on super edges, which take all possible paths between a pair of nodes, allowing the characterization of the properties of the networks within the graphs. In the proposed method, current densities of various dipoles were averaged using linear inverse problems (distributed inverse methods) and Brodmann's mapping criterion based on MRI images and EEG recordings. In the later stages, multivariate autoregressive models (MVAR) were used to estimate the frequency domain, which was then modeled by a graph. After using PCA for dimensionality reduction and decorrelation of heavily correlated measurements, each frequency band was projected into a three-dimensional space, allowing for further analysis and interpretation of the data. According to the results obtained for the dataset, the p-value yielded a value of 0.066.

In the study titled “PFT: A Novel Time-Frequency De-composition of BOLD fMRI Signals for Autism Spectrum Disorder Detection,” the authors proposed a new approach called Progressive Fourier Transform (PFT) for detecting Autism Spectrum Disorder (ASD) using fMRI signals. The authors utilized the temporal dynamics of the BOLD (blood oxygen level-dependent) data from specific brain areas for ASD categorization. The PFT was employed to derive the temporal dynamic features of the BOLD signals. This approach aimed to address the limitations of existing ASD detection systems by incorporating time-frequency components and improving feature extraction and classification. The study used the Autism Brain Imaging Data Exchange dataset for model validation, demonstrating better results with the proposed PFT model compared to existing models, including an increase in accuracy to 96.7%. This research highlights the potential of the PFT technique for analyzing rs-fMRI data from various brain diseases of the same type.

In turn, there is a need for a model for use with EEG data for disease detection.

### SUMMARY

The present disclosure provides for a forward backward Fourier transform (FBFT) model for brain disease detection.

According to one non-limiting aspect of the present disclosure, an exemplary embodiment of a forward backward Fourier transform (FBFT) model for brain disease detection.

According to a second non-limiting aspect of the present disclosure, an exemplary embodiment of a method of using a forward backward Fourier transform (FBFT) model for brain disease detection.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. In addition, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a flowchart of the proposed model for brain disorders classification from EEG signals, according to an example embodiment of the present disclosure.

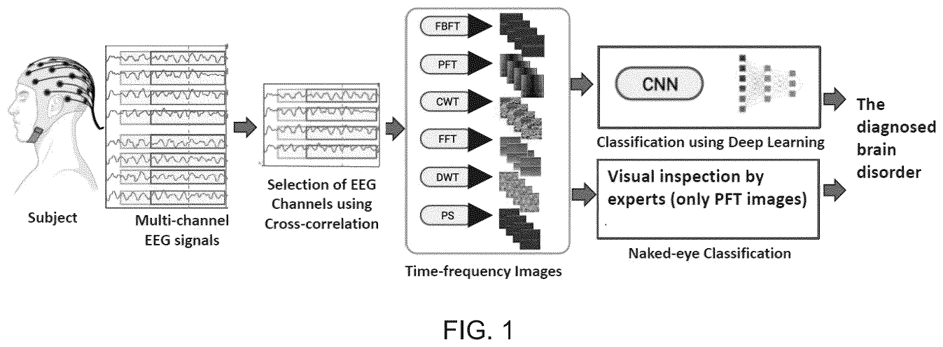

FIG. 2 shows an illustration of the Sliding Window: window size: 500 points; overlapping: 250 points, according to an example embodiment of the present disclosure.

FIG. 3 shows a correlation Matrix for two EEG from (a) Seizure, (b) non-seizure subjects and the mean correlation values of each channel, according to an example embodiment of the present disclosure.

FIG. 4 shows scalograms: (a) RGB, (b) Stitched, according to an example embodiment of the present disclosure.

FIG. 5 shows FBFT on a combined signal with frequencies 5, 10, and 30, according to an example embodiment of the present disclosure.

FIG. 6 shows a plot showing the CWT, DWT, FFT, PS, PFT, and FBFT on a combined signal, according to an example embodiment of the present disclosure.

FIG. 7 shows Convolutional Neural Network architecture, according to an example embodiment of the present disclosure.

FIG. 8 shows accuracy comparison between different methods, according to an example embodiment of the present disclosure.

FIG. 9 shows epilepsy results, according to an example embodiment of the present disclosure.

FIG. 10 shows stress results, according to an example embodiment of the present disclosure.

FIG. 11 shows Alzheimer's results, according to an example embodiment of the present disclosure.

FIG. 12 shows murmur results, according to an example embodiment of the present disclosure.

FIG. 13 shows CHBMIT SOTA comparison, according to an example embodiment of the present disclosure.

FIG. 14 shows Alzheimer's SOTA comparison, according to an example embodiment of the present disclosure.

FIG. 15 shows murmur SOTA Comparison, according to an example embodiment of the present disclosure.

FIG. 16 shows stress SOTA comparison, according to an example embodiment of the present disclosure.

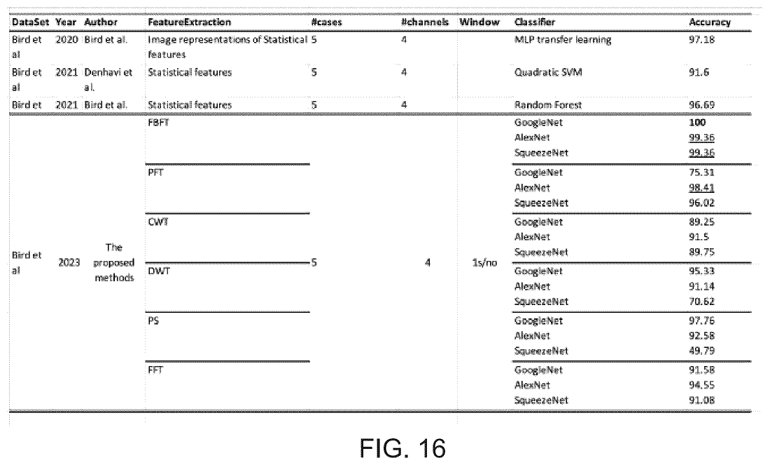

FIG. 17 shows stress SOTA comparison, according to an example embodiment of the present disclosure.

FIG. 18 shows confusion Matrix-FBFT, according to an example embodiment of the present disclosure.

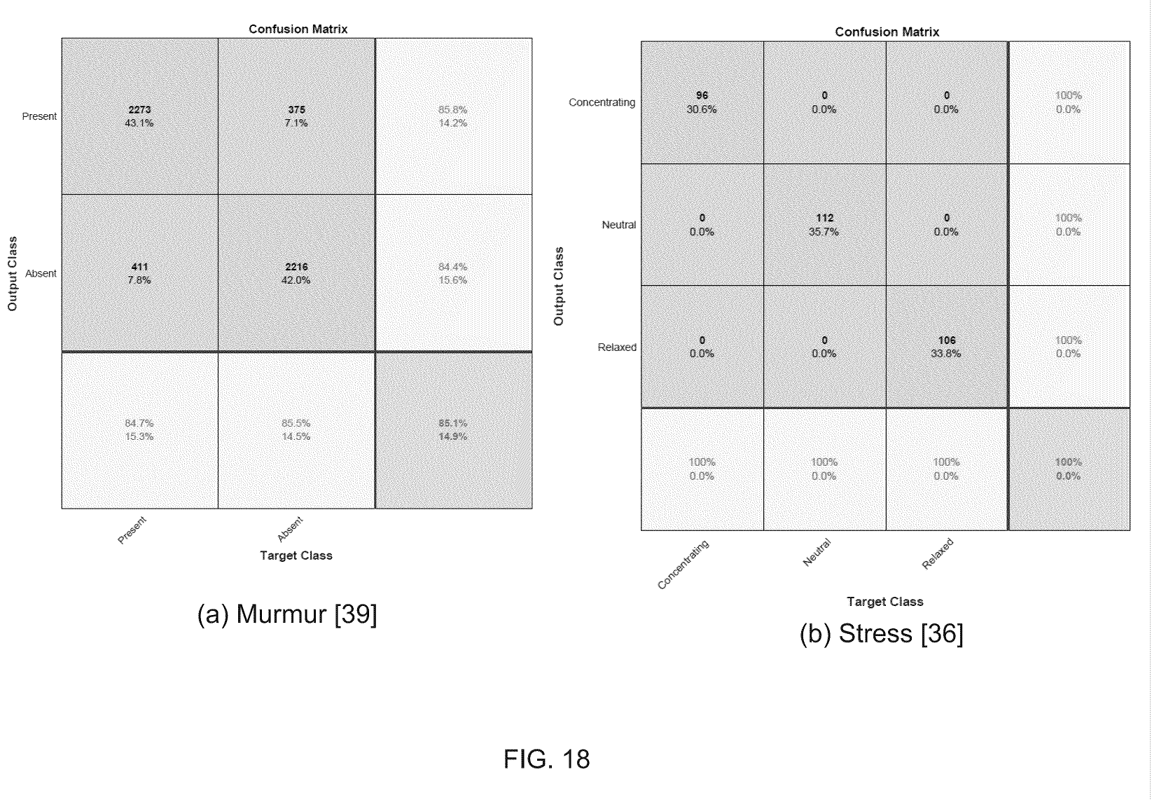

FIG. 19 shows confusion Matrix-FBFT, according to an example embodiment of the present disclosure.

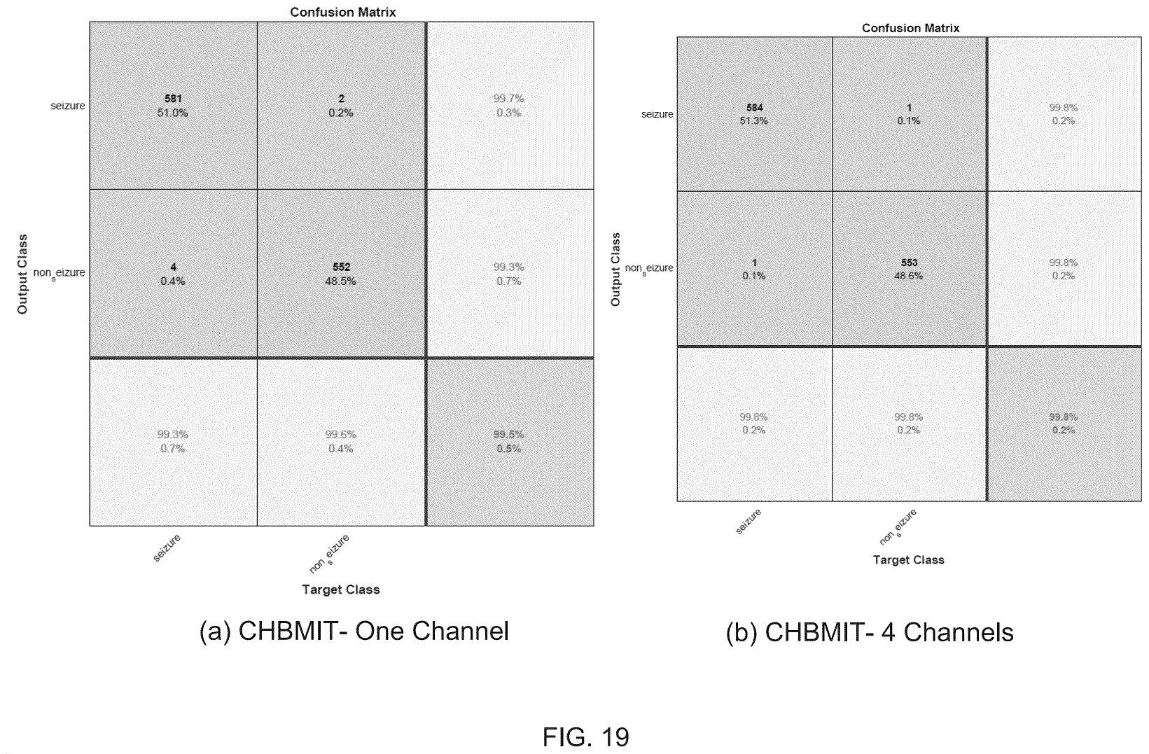

FIG. 20 shows confusion Matrix-FBFT-Alzheimer's, according to an example embodiment of the present disclosure.

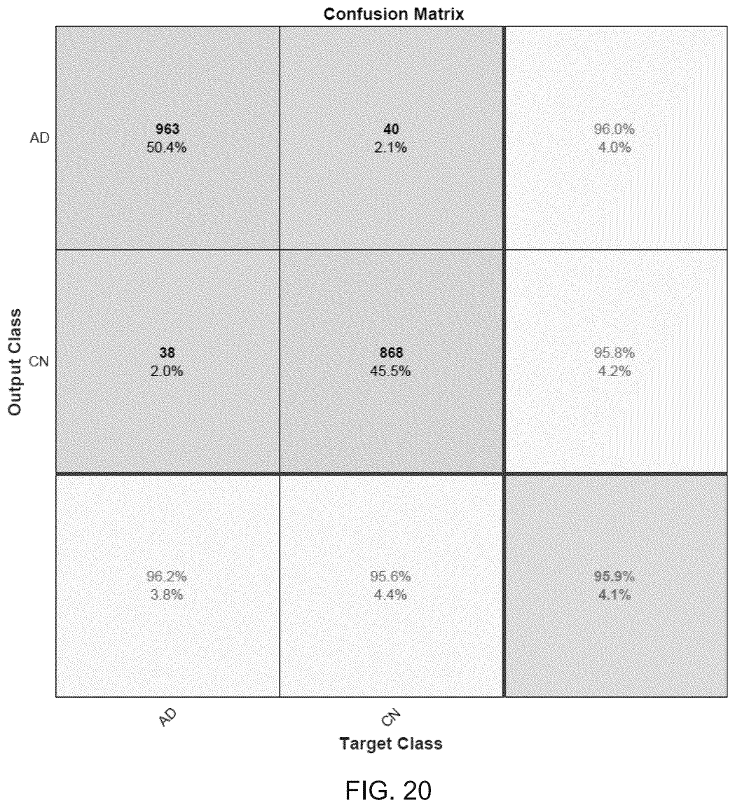

FIG. 21 shows voting Threshold:2, according to an example embodiment of the present disclosure.

FIG. 22 shows voting Threshold:5, according to an example embodiment of the present disclosure.

FIG. 23 shows eye classification, according to an example embodiment of the present disclosure.

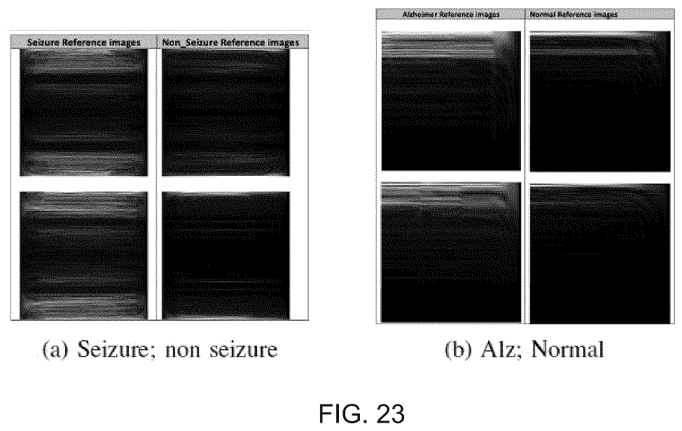

FIGS. 24A and 24B shows (a) stress, non-stress eye classification, and (b) murmur, non-murmur eye classification, according to an example embodiment of the present disclosure.

FIG. 25 shows sample images for Bird et al's Stress dataset generated by Method-1-using concatenating EEG channels' images, according to an example embodiment of the present disclosure.

### DETAILED DESCRIPTION

The present disclosure generally relates to a forward backward Fourier transform (FBFT) model for brain disease detection.

FBFT has demonstrated exceptional accuracy, surpassing state-of-the-art models in EEG-based disease classification. Within a CHBMIT dataset, FBFT achieved remarkable accuracy rates, reaching 100% when utilizing specific channel combinations for feature extraction. Even with a reduced set of channels to just one, FBFT consistently achieved high accuracies, further validating its effectiveness. In an ALZ dataset, FBFT also demonstrated superior performance in disease detection, achieving accuracies of 95.91% for AD/CN classification using GoogleNet, highlighting FBFT's potential for accurately diagnosing Alzheimer's disease and related disorders.

The consistent outstanding accuracy rates achieved by FBFT demonstrate its potential as a leading technique for EEG-based disease detection. Its superior performance suggests that it can contribute to more accurate diagnoses, ultimately leading to improved patient outcomes and more effective treatment strategies. This research showcases the promise of FBFT in advancing the field of EEG-based disease diagnosis.

Overall, FBFT offers a novel and highly effective approach to EEG-based disease detection, with the potential to revolutionize medical diagnoses and significantly enhance patient care. Additionally, FBFT achieved 100% accuracy for a Stress dataset and an accuracy of 85.1% for a murmur dataset, further highlighting its versatility and potential impact across different applications.

This disclosure explores the challenging task of translating complex visual EEG signals into a format that can be easily understood and interpreted by medical professionals as well as deep learning algorithms. It presents a comprehensive study on enhancing the interpretability of complex EEG signals in medical image classification. In the field of image classification, there exists a wide spectrum of visual representations that can be difficult for doctors to comprehend, hindering their ability to make accurate diagnoses. This disclosure leverages the power of FBFT to extract meaningful features and convert EEG signals to medical images. Additionally, this disclosure introduces the concept of eye naked classification, which involves integrating domain-specific knowledge and clinical expertise into the classification process. By combining these approaches, this disclosure demonstrates the potential to improve the interpretability of visual signals and empower medical professionals with a deeper understanding of the underlying information contained within medical images. This naked-eye interpretation combined with the deep learning-based brain disorders classification has achieved better results. The main contributions of this disclosure are as follows:
Enhancing EEG Signal Interpretability: Transforming to 2D Images using a novel time-frequency feature extraction technique Forward Backward Fourier Transform (FBFT) for Medical Image Classification.
Optimal EEG Channel Selection for Improved Classification: A Time-Saving Cross-Correlation Approach.
Fusion of Selected EEG Channels: Creating a Comprehensive Time-Frequency Scalogram Image for ML/DL Disease Classification.
Comparative Analysis of Feature Extraction Methods for Brain Disorder Diagnosis: A Focus on FBFT and Deep Learning Models.
Naked-Eye Diagnosis of Brain Disorders: Leveraging FBFT-Generated Time-Frequency Images from EEG Signals.

Several deep learning methods have been proposed for the classification of brain disorders. However, none of these methods have analyzed models using different time-frequency transformed EEG images. Moreover, most of these approaches concentrate solely on diagnosing a single brain disorder based on EEG signals. This disclosure introduces a paradigm shift by not only revolutionizing the visual interpretation and representation of EEG data for neurologists but also by providing deep learning models with easily distinguishable input images for various brain disorder classes. This advancement enhances diagnostic accuracy, facilitates optimized treatment planning, and ultimately leads to improved patient outcomes.

### Experimental Data

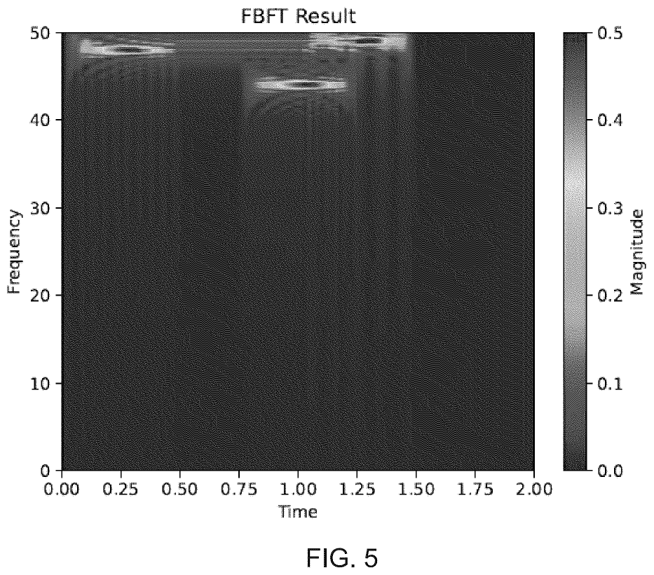

Epilepsy—Data Acquisition: Data were collected from the EEGs of children at the Children's Hospital Boston (CHB) and at the Massachusetts Institute of Technology (MIT). The data are publicly accessible and are available on the website Physionet.org. A total of 916 hours of EEG signals were recorded from 22 pediatric participants with intractable seizures for a total of one hour or four hours. Five males and 17 females participated in the study, ranging in age from 3-22 years and 1.5-19 years, respectively. The number of electrodes varied between 23 to 28 electrodes for different patients. This dataset was sampled at a rate of 256 Hz, with 23 EEG signals per file, and 198 seizures were annotated with their beginning and end times. There are 23 channels in most records, with a few having 24 and 26; FIG. 5 shows seizures and non-seizures records. The files can be downloaded as ZIP files (42.6 GB) using European Data Format (.edf), which can be accessed via a terminal or Google Cloud Storage Browser. Preictal and interictal labels were not included in this data set, but could be extracted from the meta-data files for each patient.

Stress—Data Acquisition: A dataset compiled by literature was used to train and evaluate the different approaches proposed in this disclosure. Literature used a Muse headband equipped with four dry EEG sensors (TP9, AF7, AF8, and TP10) to record EEG signals for three mental states: relaxed, neutral, and concentrating, while exposing subjects to corresponding stimuli. The three mental states were utilized as class labels for the classification task. The dataset consists of 25 Excel sheets of raw EEG data recorded by five participants over two-minute sessions for each of the three mental states and is available online.

Alzheimer—Data Acquisition: Alzheimer's disease (AD) is a neurodegenerative disorder, which causes memory loss, changes in behavior, and other cognitive problems. It is most common in people over 65, but can occur at younger ages. Recently a new dataset of EEG signals for Alzheimer is developed by the literature. The EEG dataset has signal acquired for 88 subjects who were resting with their eyes closed. Of these, 36 were AD patients, 23 with frontotemporal dementia (FTD), and 29 with cognitive normal. The neurological state of each participant was evaluated using a test called Mini Mental State Examination (MMSE), a standardized test that scores cognitive decline from 0 to 30, where 0 is for more severe cases.

Murmur Data Acquisition: The dataset utilized was gathered from two screening campaigns carried out in Northeast Brazil during specific time frames: one in July/August 2014 and another in June/July 2015. This dataset comprises recordings of heart sounds, with varying durations ranging from 5 to 45 seconds. Additionally, the dataset includes demographic information such as age groups, gender, height, weight, and pregnancy status. In total, the dataset encompasses 1568 patients, with 60% of them (942) designated for participant training. Each patient may have provided up to six heart sound recordings, resulting in a comprehensive dataset containing 5272 recordings, with 3163 recordings assigned to the training set. These recordings are categorized based on the location of capture, including pulmonary valve, aortic valve, mitral valve, tricuspid valve, or other. Furthermore, each patient is associated with a heart murmur label, which can be categorized as present, unknown, or absent.

### Disclosed Technology: Preprocessing

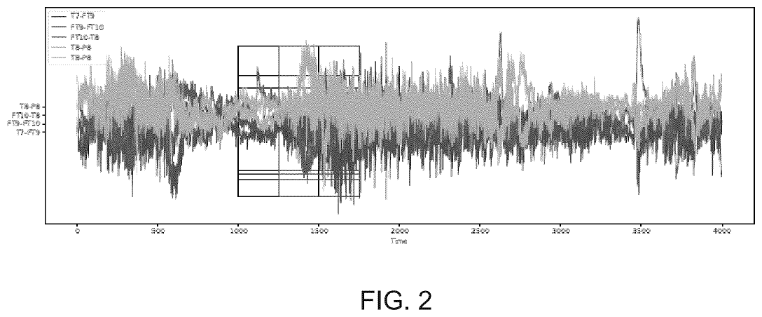

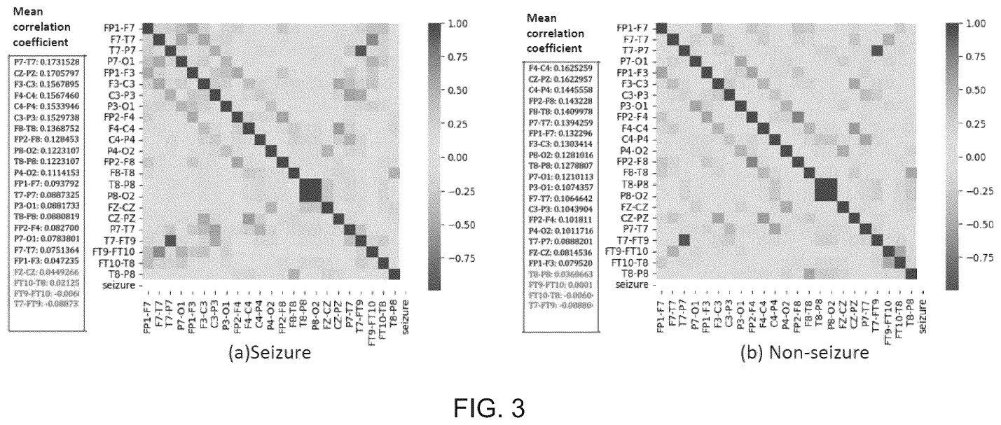

Sliding Window size selection: The sliding window technique is commonly used for feature extraction in EEG signals because of the complexity, nonlinearity, and nonstationary of these signals. These characteristics make it difficult to extract meaningful information from the signals using traditional methods. The sliding window method works by dividing the EEG signal into overlapping windows, each of which is considered stationary for the duration of the window. Features are then extracted from each window, allowing for a more accurate representation of the signal's characteristics. The size of the window is determined by the cross-correlations between the EEG channels, which are used to determine the optimal size of the window based on the temporal distribution of the signal within a specific time window or at specific data points over time, illustrated in FIG. 2 der diagnosis are typically multi-channel, but many channels contain redundant signals, increasing complexity and memory usage. Instead of using all channels, it is common practice to select a subset for efficiency. This disclosure introduces a novel method based on channel correlation. This disclosure creates spatial images for each EEG signal, adjust their temporal distribution, and extract features. Using cross-correlation, this disclosure identifies the most suitable channels for each disease, optimizing accuracy and informativeness, FIG. 3 First; this disclosure computes the correlation coefficient between two EEG channels X1 and X2 as follows.

Channel selection: EEG datasets used for brain disorder diagnosis are typically multi-channel, but many channels contain redundant signals, increasing complexity and memory usage. Instead of using all channels, it is common practice to select a subset for efficiency. This disclosure introduces a novel method based on channel correlation. This disclosure creates spatial images for each EEG signal, adjust their temporal distribution, and extract features. Using cross-correlation, this disclosure identifies the most suitable channels for each disease, optimizing accuracy and informativeness, FIG. 3 First, this disclosure computes the correlation coefficient between two EEG channels X1 and X2 as follows:

$${r}_{X 1 X 2} = \frac{{\sum }_{i = 1}^{n} ( {x}_{1 i} - {\overline{x}}_{1} ) ( {x}_{2 i} - {\overline{x}}_{2} )}{\sqrt{{\sum }_{i = 1}^{n} {( {x}_{1 i} - {\overline{x}}_{1} )}^{2} {\sum }_{i = 1}^{n} {( {x}_{2 i} - {\overline{x}}_{2} )}^{2}}} \qquad ( 1 )$$

Where n is the total number of samples, x1 and x2 are the mean and x1i, x2i are the samples of the two channels. This correlation coefficient is computed for each channels with all other channels resulting in a correlation matrix (CorrMat) given as follows:

$$CorrMat = [ \begin{matrix}{r}_{X 1 X 1} & {r}_{X 1 X 2} & ⋯ & {r}_{X 1 Xn} \\ {r}_{X 2 X 1} & {r}_{X 2 X 2} & ⋯ & {r}_{X 2 Xn} \\ . & & & \\ . & & & \\ {r}_{XnX 1} & {r}_{XnX 2} & ⋯ & {r}_{XnXn}\end{matrix} ] \qquad ( 2 )$$

The mean of each column of the CorrMat is then computed to get a single value, which is the mean correlation coefficient for each channel. The examples of the CorrMat and mean values are computed for two randomly selected subjects with seizure and non-seizure are shown in FIG. 3.

The channels with low mean correlation coefficient values are selected. This is because highly correlated channels tend to capture redundant information, which can increase noise and reduce the accuracy of the classification algorithm. In contrast, uncorrelated channels provide unique information and can complement each other, leading to more accurate results. For the two subjects as shown in FIG. 3, the channels that have the least mean correlation coefficient are T8-P8, FT9-FT10, FT10-T8, T7-FT9 for seizure and FZ-CZ, FT9-FT10, FT10-T8, T7-FT9 for non-seizure. These channels are selected for further feature extraction.

### Feature Extraction

This disclosure employed a variety of established feature extraction techniques, including Fast Fourier Transformation (FFT), Power Spectrum (PS), Continuous Wavelet Transformation (CWT), Discrete Wavelet Transform (DWT), and Progressive Fourier Transform (PFT), alongside this disclosure's innovative FBFT method. These techniques were selected to create images from EEG data for a comparative analysis with this disclosure's novel method. By leveraging these well-established feature extraction methods, one can gauge the effectiveness and performance of this disclosure's novel approach in relation to widely recognized techniques. This comparative assessment provides a holistic evaluation of the novel method's efficiency and potential benefits when contrasted with existing methodologies.

Creating Scalograms: The spectrogram is a graphical representation that depicts the strength of a signal at different frequencies over time within a waveform. It is widely recognized as one of the most commonly used tools in signal analysis, as it provides a visualization of the frequency distribution of a signal in the time domain. Similarly, the scalogram demonstrates the correlation between the signal and the scaled wavelet across time, which can be mathematically expressed as |XWT (a, b)|2.

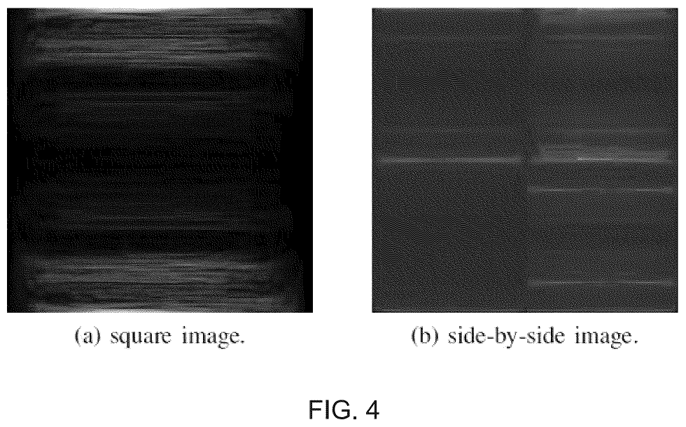

This disclosure employed two methods, namely RGB and concatenating images, to generate scalograms or spectrograms for each time-framed window of EEG data as shown in FIG. 4.

Using Fast Fourier Transform (FFT): The fast Fourier transform (FFT) algorithm is frequently used to calculate Discrete Fourier Transform (DFT) that is used to analyze signals in the frequency domain. The DFT is calculated at discrete frequencies fn=n, where n=0, 1, 2, . . . , N−1 using the equation:

$$F ( k ) = \frac{1}{N} {\sum }_{k = 0}^{N - 1} x ( n ) {e}^{- j 2 π nk / N} \qquad ( 3 )$$

DFT consumes O N2 multiplies and adds for large signals, whereas the FFT only considers sample point intervals N as a power of two N=2m, m∈N and consumes O(N log N) multiplies and adds.

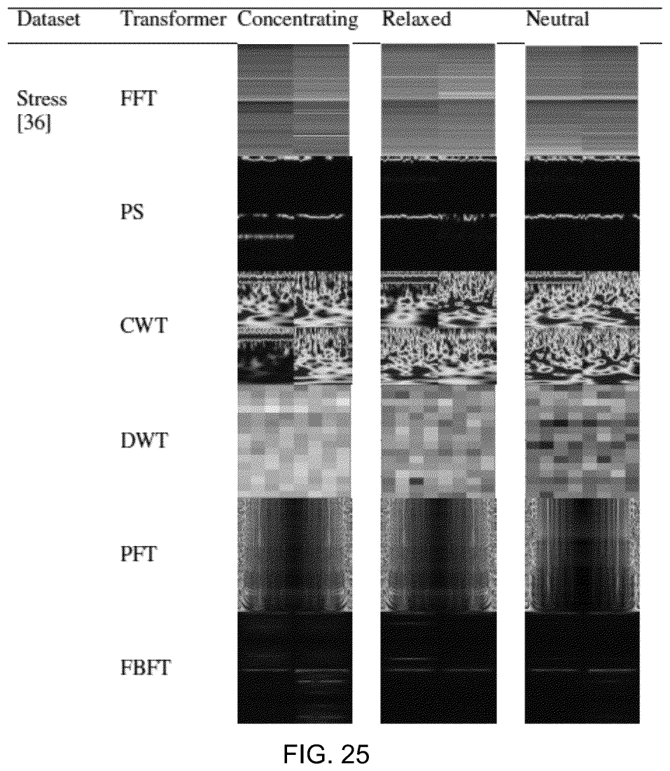

In this method, this disclosure used FFT to convert EEG signals from the time domain to the frequency domain, creating spatial images or spectrograms, as shown in FIG. 25.

Using the Power Spectrum: A power spectral density (PSD) is also known as a spectral density or power spectrum, which can be calculated with the Fourier transform and the FFT algorithm. The latter shows how the strength of a random signal varies with frequency, i.e., it shows the frequency do-main distribution of the signal's intensity. PSD is the squared absolute value of the Fourier transform with an equation:

$$S ( {f}_{n} ) = \frac{1}{N} {\vert {\sum }_{k = 0}^{N - 1} x ( n ) {e}^{- j 2 π nk / N} \vert}^{2} \qquad ( 4 )$$

The Power Spectrum (PS) was used as a second method. The resulting spectrogram displayed energy concentrations at specific frequencies and times, FIG. 25.

Using Continuous Wavelet Transform (CWT): The CWT analyzes a signal by using long low-frequency windows and short high-frequency windows using the equation:

$${X}_{WT} ( a , b ) = \frac{1}{\sqrt{\vert a \vert}} {\int }_{- ∞}^{∞} x ( t ) {ψ}^{*} ( \frac{t - b}{a} ) dt \qquad ( 5 )$$

Where * is conjugate in complex number, ψ(t) is the mother wavelet, and a and b are Wavelet coefficients.

A CWT wavelet coefficient is associated with a scale (frequency) and a point in the time domain. Continuous mother wavelets are scaled by factor a and translated by factor b; thus, the wavelet can be moved across the signal by adjusting b, while changing scale ‘a’ affects the frequency and window length of the wavelet. As the value of a decreases, the wavelet will appear more compressed, and high-frequency data can be captured. Increasing the value of a, on the other hand, will extend the wavelet and collect low-frequency information. The wavelet shifts to the left as b is decreased and to the right as b is increased. As the scaling and translating variables have continuous values, the results of wavelet analysis are denoted by scale rather than frequency.

The CWT provided a wealth of frequency-time information as images or scalograms displaying the absolute value of a signal's CWT coefficients. In non-stationary signal analysis, the Morlet wavelet was used since it provides good time and frequency resolution as well as the spatiotemporal map of the signal, FIG. 25.

Using Discrete Wavelet Transform (DWT): In this method, the Discrete Wavelet Transformation (DWT) is utilized both as a feature extraction technique and a signal-to-image transformer. To perform the DWT, the Daubechies wavelet with a mother wavelet of db4 is specifically chosen. This wavelet has been found to exhibit exceptional accuracy in classifying non-stationary data, such as EEG signals, as evidenced by recent studies. This is expressed as

$${ψ}_{i , k} ( u ) = {2}^{- i / 2} ψ ( {2}^{- i} u - k )$$

follows:

The coefficients are obtained using the following expression:

$${W}_{i , k} = W ( {2}^{i} , k {2}^{i} ) = {2}^{- i / 2} {\int }_{- ∞}^{∞} f ( u ) \overline{ψ ( {2}^{- i} u - k )} du$$

In the process, the detailed coefficients or frequency values of each EEG time frame window are first computed alongside the approximation coefficients. These two sets of coefficients are then averaged together. Subsequently, the resulting averaged values are further averaged with the coefficients obtained from adjacent EEG channels. By obtaining these averaged values, three metrics are derived. These metrics are then represented as RGB images for each analyzed time frame, allowing for a visual representation of the data and facilitating further analysis.

4) Using Progressive Fourier Transform (PFT): The Progressive Fourier Transform (PFT) is a time-frequency representation that builds upon the Fourier Transform. It utilizes the sliding window technique to progressively compute Fourier transforms of signal values. This technique allows for the exclusion of out-of-range signal values by employing the equation:

$$F ( f , S ) = {\int }_{- ∞}^{∞} f ( x ) {1}_{x \leq S} {e}^{- j 2 π τ} d τ , S ∈ R \qquad ( 6 )$$

5) Using Forward Backward Fourier Transform: The novel Forward-Backward Fourier Transform (FBFT) process involves forward and backward transformations, where the signal is divided into subarrays, undergoes zero padding, and Fast Fourier Transform (FFT) operations. This process allows for the extraction of crucial spectral information from EEG signals. The maximum magnitude values obtained from both transformations are carefully analyzed. To improve the FBFT representation, a transposition and modification step is applied, resulting in an altered representation. At the same time, the absolute values of the original EEG signal (A) are computed. The final FBFT representation is generated by calculating the element-wise minimum between the two transforms. This comprehensive methodology enables the extraction of crucial spectral and time-varying features from EEG signals, pro-viding valuable insights for disease diagnosis and analysis, illustration of a FBFT on a combined signal given in FIG. 5.

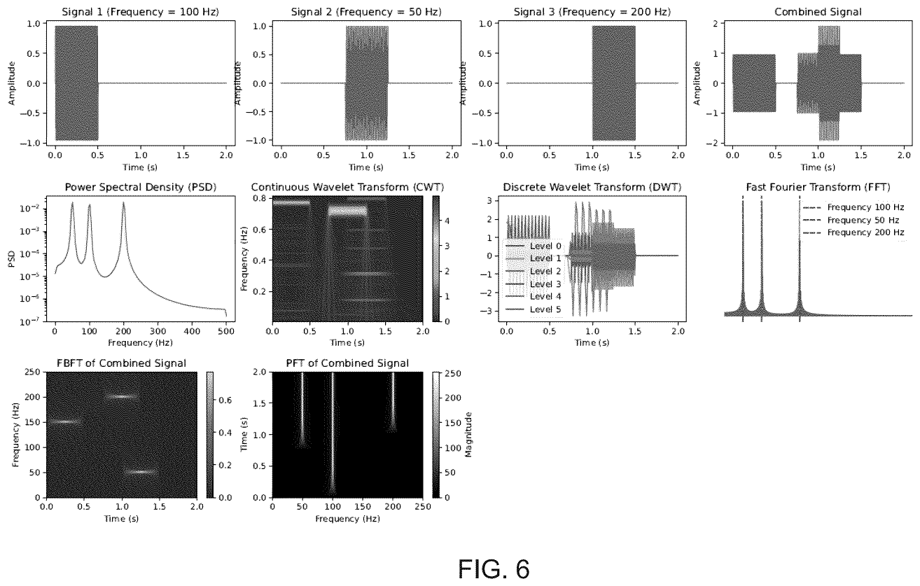

FIG. 6 illustrates a comparative analysis between FBFT and traditional techniques such as FFT, CWT, and DWT when applied to a composite signal with known frequency components. FBFT stands out due to its impressive capability to achieve precise time-frequency localization. In contrast, while FFT provides frequency information, it lacks the precision to pinpoint these frequency components in the time domain precisely. Similarly, although CWT and DWT offer improved time-frequency resolution compared to FFT, they may still have limitations in capturing subtle shifts in the signal.

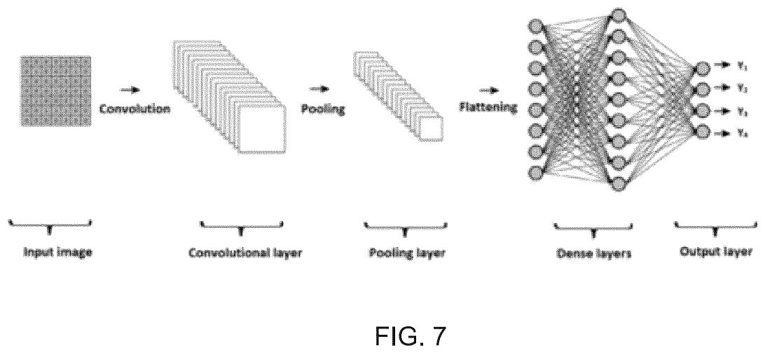

Classification Models—1) convolutional neural networks: A CNN model with six convolution layers was developed for classifying mental states in various datasets. CNNs were chosen for their ability to learn abstract features, efficiently classify images, overcome limitations in other algorithms, and reduce parameter amounts using dimensional reduction methods. The fully connected layers receive input from the pooling layers and output a feature vector representing high-level features of the input image. The pooling layer reduces the feature map size through downsampling, using max or average pooling approaches. The flattened feature map is then passed to the out-put layer for classification. Transfer learning with pretrained models, such as ResNet, GoogLeNet, and AlexNet, has been widely used to improve accuracy and speed up training in CNN-based applications, FIG. 7.

The following metrics are used to evaluate the performance of this disclosure's model:

$$ACC = \frac{T P + T N}{T P + F P + F N + T N} \qquad ( 7 )$$

$$Precision = \frac{T P}{T P + F P} \qquad ( 8 )$$

$$Recall = \frac{T P}{T P + F N} \qquad ( 9 )$$

$$Specificity = \frac{T N}{T N + F P} \qquad ( 10 )$$

$$F1\_score = \frac{2 \cdot T P}{2 \cdot T P + F P + F N} \qquad ( 11 )$$

where:
   TP: True Positives
   TN: True Negatives
   FP: False Positives
   FN: False Negatives

### Results and Discussions

Below is a comprehensive comparison of the FBFT (Feature-Based Frequency Transformation) model with other feature extraction models that have been studied in the context of this research. This disclosure's comparison focuses on evaluating the performance of FBFT based on various factors such as channel selection, window size, and overlap selection for each specific disease.

Evaluation of FBFT Performance—to assess the effectiveness of FBFT, this disclosure analyzes its performance based on different parameters. Firstly, this disclosure examines the impact of channel selection on the accuracy of the model. Next, this disclosure investigates the influence of window size and overlap selection on the performance of FBFT.

Comparing Accuracy with State-of-the-Art Models—to further validate the effectiveness of FBFT, this disclosure compares its accuracy in disease detection with the results obtained by other models in the state of the art.

### Comparison with Other Models

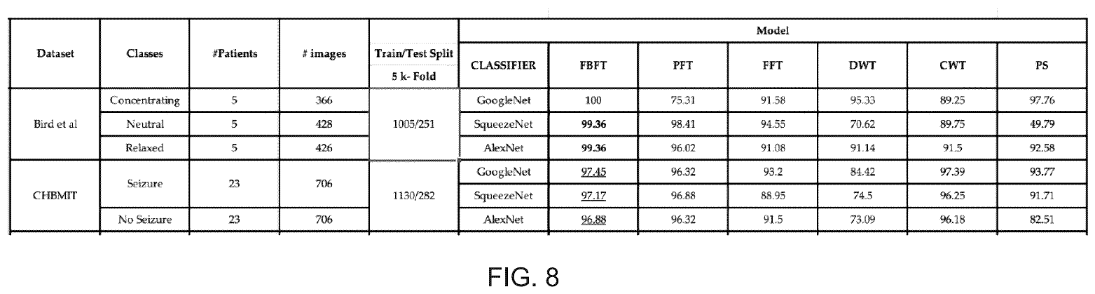

This disclosure's model was initially assessed using various transformers including PFT, FFT, DWT, CWT, and Ps. The experimental results indicate that FBFT demonstrates superior performance compared to the other transformers when applied to Google Net, Squeezenet, Alex Net on literature's stress dataset and CHBMIT's epilepsy dataset, FIG. 8.

To evaluate the performance of FBFT, different classifiers were employed, namely GoogleNet, AlexNet, and SqueezeNet. The reported accuracies for disease classification using FBFT were 100% for GoogleNet, 99.36% for AlexNet, and 99.36% for SqueezeNet.

In comparison with other proposed methods, FBFT demonstrated significant improvements in accuracy. For instance, when comparing FBFT with PFT using the same classifiers, FBFT achieved higher accuracies across the board. The ac-curacies for disease classification using PFT with GoogleNet, AlexNet, and SqueezeNet were 75.31%, 98.41%, and 96.02%, respectively.

Similarly, when comparing FBFT with other transformations such as CWT (Continuous Wavelet Transform), DWT (Discrete Wavelet Transform), PS (Power Spectrum), and FFT (Fast Fourier Transform), FBFT consistently outperformed these methods in terms of accuracy. The specific accuracies for disease classification using FBFT, CWT, DWT, PS, and FFT with the three classifiers are illustrated in FIG. 8.

Overall, the results indicate that the proposed FBFT method consistently outperformed alternative transformations in dis-ease classification. The higher accuracies achieved by FBFT suggest that this feature-based frequency transformation approach is effective in capturing relevant information from the data and accurately distinguishing between different disease conditions.

### Evaluation of FBFT Performance

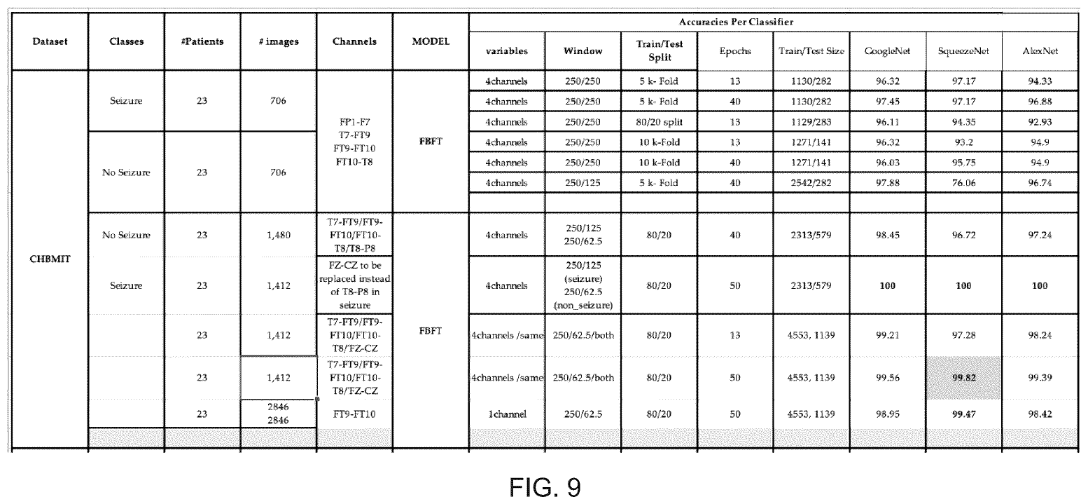

In the CHBMIT dataset, FIG. 9, this disclosure experimented with various channel combinations, window sizes, and training splitting techniques. Among these, the most promising results were achieved by selecting channels based on this disclosure's suggested method utilizing cross-correlation. These selected channels, namely T7-FT9/FT9-FT10, FT10-T8, and FZ-CZ, yielded an impressive accuracy of 99.82% when fed to SqueezNet. This disclosure found that employing an optimal window size of 250 points with a 62.5 points overlap further enhanced the performance.

Additionally, this disclosure obtained notably high accuracy using a single channel, FT9-FT10, achieving an accuracy of 99.47% with the SqueezNet classifier.

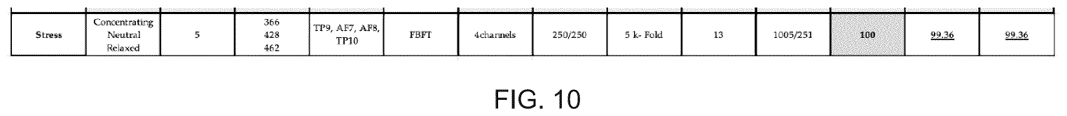

Literature's dataset consists of 4 channels. Therefore, the step of selecting specific channels was skipped in this evaluation. Surprisingly, a remarkable accuracy of 100% was achieved using Googlenet as the model, with a window size of 250 and no overlap. This outstanding result showcases the effectiveness of the FBFT with the Googlenet model in accurately classifying the data from literature's dataset without the need for channel selection or overlapping windows, FIG. 10.

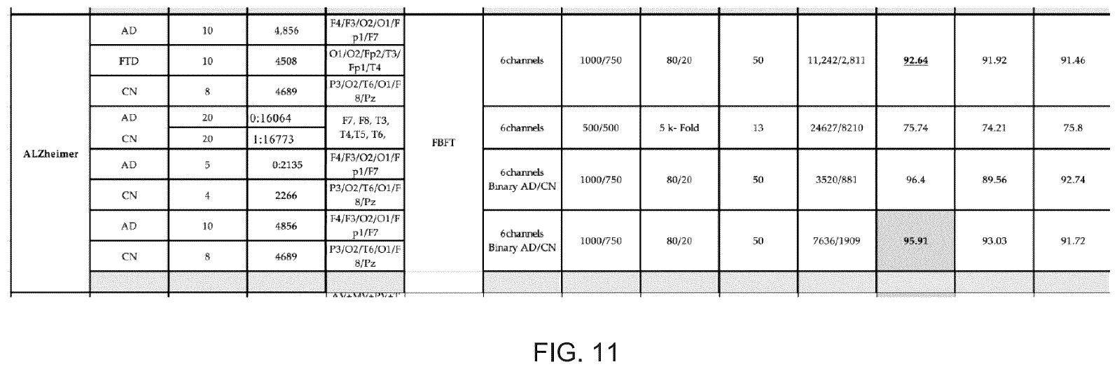

In the context of the Alzheimer dataset, the performance was not as strong when utilizing all four channels. However, by incorporating six channels and specifically focusing on the binary classification of Alzheimer's Disease (AD) versus Control (CN), there was a notable improvement. The achieved accuracy reached 95.91%, demonstrating promising results. This significant enhancement was accomplished by utilizing a window size of 1000 with a 750-point overlap. These findings highlight the importance of optimizing the window size and overlap, as they can contribute to substantial advancements in accurately classifying AD versus CN within the Alzheimer dataset, FIG. 11.

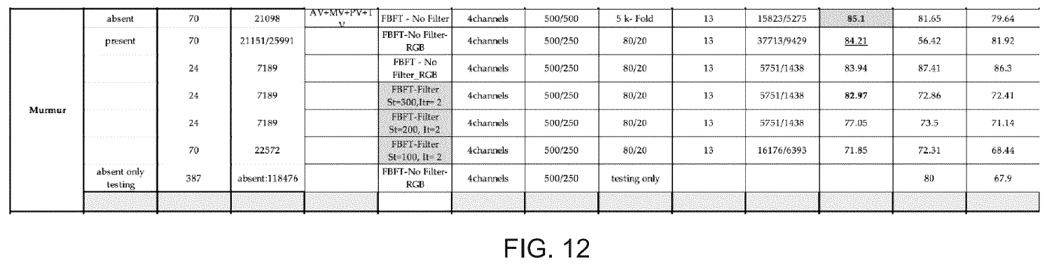

For the challenging Murmur dataset, this disclosure approached it by treating the 4 valves as separate channels. This resulted in a dataset of approximately 46,000 images. To analyze these images, this disclosure employed a window size of 500 points without any overlap. Through this approach, this disclosure achieved a notable accuracy of 85.1%, FIG. 12 that represents the best result obtained for this dataset. This outcome underscores the effectiveness of considering the valves as individual channels and optimizing the window size for accurate classification in the Murmur dataset.

### Comparison with State-of-the-Art

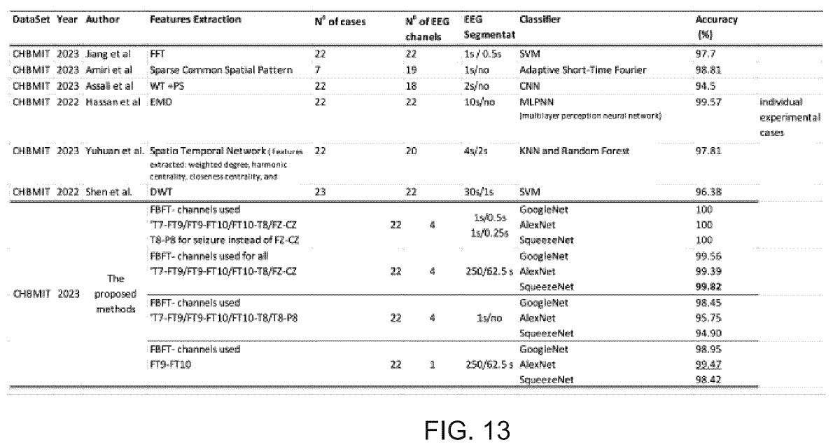

The FBFT (Feature-Based Frequency Transformation) model has demonstrated superior performance compared to several state-of-the-art models in the field of disease detection utilizing EEG data FIG. 13.

In the CHBMIT dataset, FBFT achieved remarkable accuracy rates across different configurations. When considering the use of specific channels (‘T7-FT9/FT9-FT10/FT10-T8/FZ-CZ’) for feature extraction, FBFT achieved an accuracy of 100% when utilizing GoogleNet, AlexNet, and SqueezeNet classifiers. Even when using a reduced set of channels (‘T7-FT9/FT9-FT10/FT10-T8/T8-P8’), FBFT still achieved high accuracies of 99.56%, 99.39%, and 99.82% respectively with the same classifiers.

Furthermore, FBFT's performance was also notable when focusing on specific channel pairs. For instance, when using the channel pair ‘FT9-FT10’ for feature extraction, FBFT achieved an accuracy of 98.95%, 99.47%, and 98.42% respectively with GoogleNet, AlexNet, and SqueezeNet classifiers.

In comparison to other models in the state of the art, FBFT consistently outperformed them in terms of accuracy. For instance, the highest reported accuracy among other models was 99.57% by literature. using the EMD feature extraction method. However, FBFT surpassed this with its highest accuracy of 100% in various configurations.

These results demonstrate the effectiveness and superiority of the FBFT model in detecting diseases using EEG data, showcasing its potential as a leading feature extraction model in the field.

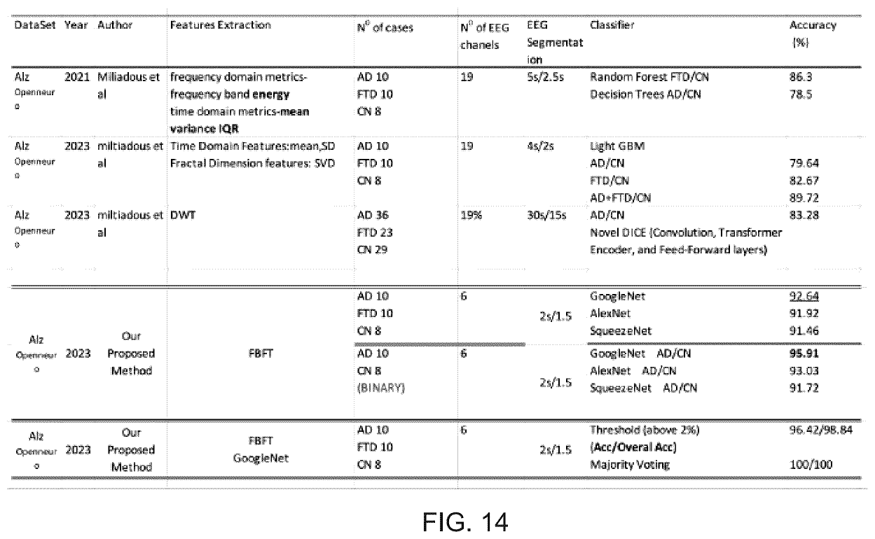

In comparison with the state of the art methods in the ALZ dataset, the proposed FBFT (Feature-Based Frequency Transformation) method demonstrated superior performance in disease detection using EEG data, FIG. 14

The study by literature compared the FBFT method with other approaches in the same dataset. The dataset consisted of 6 cases with 10 cases of AD, 10 cases of FTD, and 8 cases of CN. The EEG data was segmented using a window size of 2 seconds with a 1.5-second overlap. The classifiers used were GoogleNet, AlexNet, and SqueezeNet.

The reported accuracies for the FBFT method were 92.64%, 91.92%, and 91.46% for AD, FTD, and CN classification, respectively. In the binary classification task between AD and CN, the accuracies were 95.91% for AD/CN classification using GoogleNet, 93.03% for AD/CN classification using AlexNet, and 91.72% for AD/CN classification using SqueezeNet.

These results indicate that the FBFT method outperformed the other methods in accurately classifying the different dis-ease categories in the ALZ dataset. The higher accuracies achieved by the FBFT method suggest that the proposed feature-based frequency transformation approach is effective in capturing relevant information from the EEG data and distinguishing between different disease conditions.

The improved performance of the FBFT method highlights its potential as a promising technique for disease detection in Alzheimer's disease and related disorders. Its ability to outperform state-of-the-art methods indicates that it can contribute to more accurate and reliable diagnoses, leading to better patient outcomes and more effective treatment strategies.

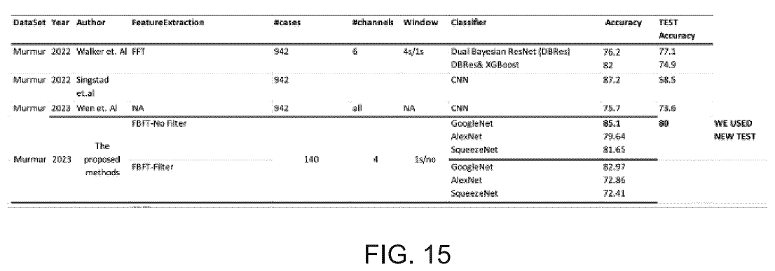

The performance of FBFT was evaluated using different classifiers, specifically GoogleNet, AlexNet, and SqueezeNet. The reported accuracies for murmur classification using FBFT were 85.1% for GoogleNet, 79.64% for AlexNet, and 81.65% for SqueezeNet, FIG. 15.

It is important to note that the literature used the FFT (Fast Fourier Transform) for feature extraction. Their proposed method, Dual Bayesian ResNet (DBRes) combined with XGBoost, achieved an accuracy of 76.2%. In comparison, FBFT outperformed this method with an accuracy of 85.1% using GoogleNet.

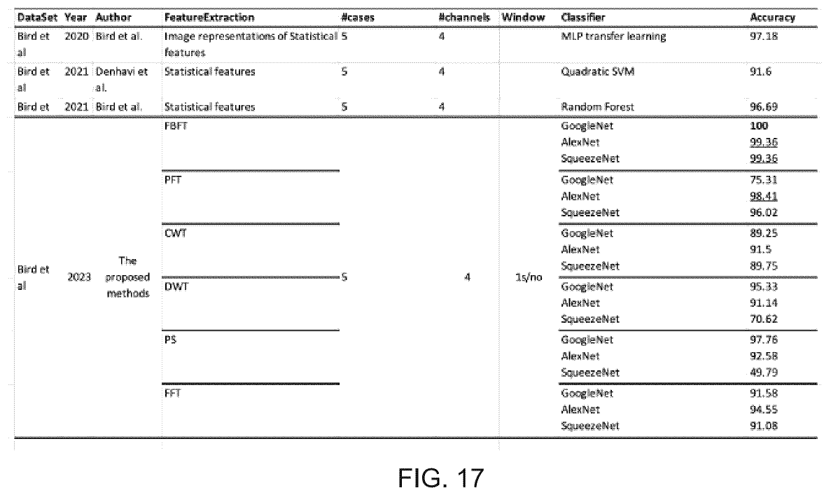

Overall, the results suggest that the proposed FBFT method, especially without a filter, shows promise in murmur classification, outperforming some state-of-the-art methods that used FFT. The higher accuracies achieved by FBFT indicate its potential for accurately detecting and classifying murmurs in the provided dataset. For the stress dataset, FBFT achieved the highest accuracy of 100% using the Googlenet classifier. FIG. 17

### Classification by Voting

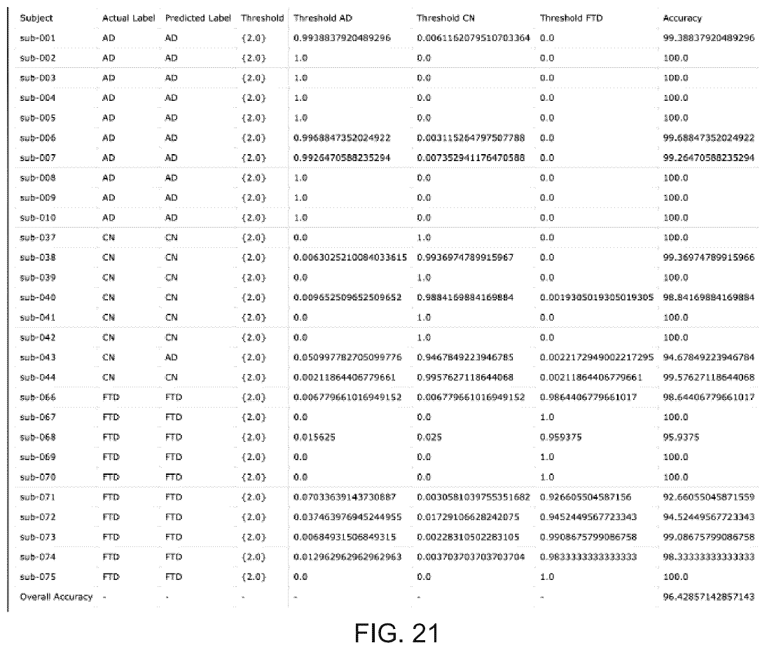

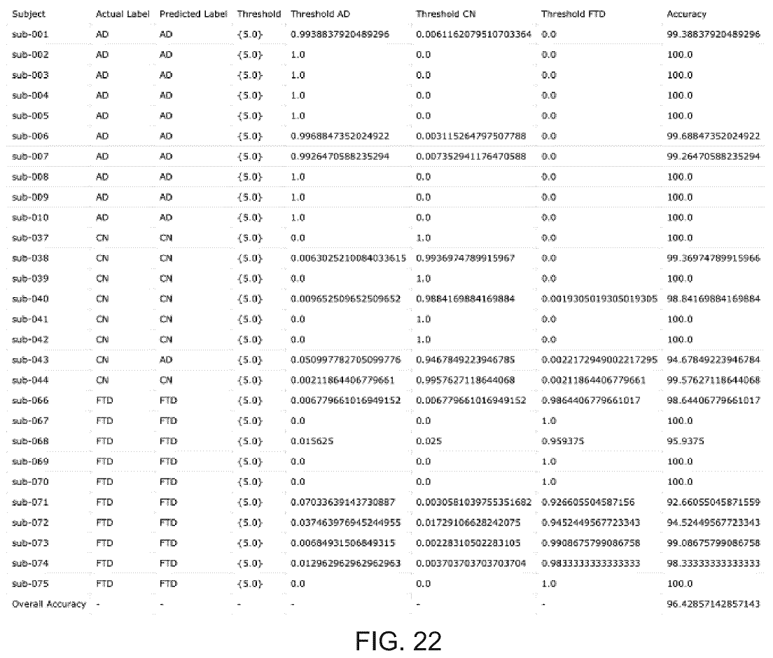

The proposed method of classification by voting introduces a new approach to determine the predicted label for a subject. In this method, the occurrences of different labels such as (AD, CN, and FTD) are counted for the subject. The counts are then used to calculate the probabilities of each label. The method has also surpassed another main method, where GoogleNet, AlexNet, and SqueezeNet achieved accuracies of 95.91%, 93.03%, and 91.72%, respectively. This demonstrates the robustness and superiority of the classification by voting approach, even when compared to well-established deep learning models, illustrated in FIGS. 21 to 22.

### Naked Eye Classification

The survey conducted for this research study, part of this research study will focus on the naked-eye classification of brain diseases using EEG signals and serves as an integral component of the larger field of neuroscience AI. The main objective is to advance the understanding and detection of brain diseases through visual analysis.

Participants are kindly invited to take part in a brief survey, which typically takes 3-7 minutes to complete. The survey entails examining a series of reference images that represent various brain diseases such as epilepsy, Alzheimer's, stress, and murmur (heart sound). Participants will be asked to visually evaluate the images and determine if they appear normal or abnormal based on their personal judgment.

The primary purpose of this survey is to gather valuable in-put and insights from participants. This input will significantly contribute to developing a practical approach for healthcare practitioners to detect brain and heart diseases through visual analysis. It is crucial to emphasize that participation in this survey is voluntary, and all responses will be treated with the utmost confidentiality.

By participating in this survey, individuals actively support the progression of the study and make a valuable contribution to the advancement of research in this critical area. The survey comprises four sections, each addressing different aspects:
1) Demographic Questions: This section focuses on gathering personal information and background details.
2) Image Assessment—Brain Diseases/Epilepsy: In this section, participants will encounter 20 images associated with brain diseases. They will be required to answer multiple-choice questions to determine whether each image represents a normal condition or not.
3) Image Assessment—Heart Diseases/Murmur: Similar to the previous section, this section includes 20 images related to heart diseases. Participants will answer multiple-choice questions to identify if the image depicts a normal condition or not.
4) Image Assessment—Brain Diseases/Stress: The fourth section presents a set of 20 images associated with brain diseases. Participants will once again answer multiple-choice questions to determine if the image represents a normal condition or not.
5) Image Assessment—Brain Diseases/Alzheimer: The final section showcases the last set of 20 images pertaining to brain diseases. Participants will answer multiple-choice questions to determine if the image represents a normal condition or not.

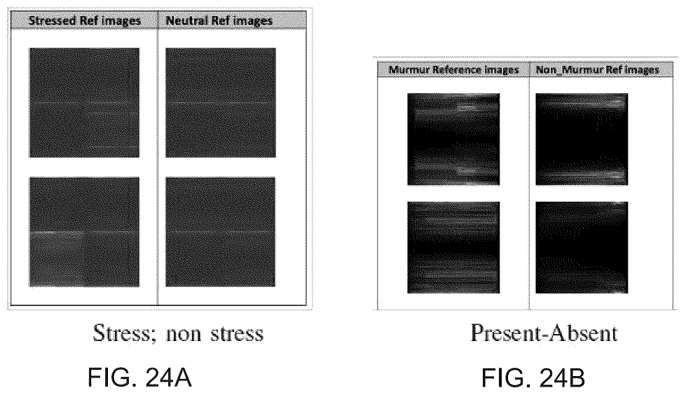

Upon completion of the survey, participants can submit their responses. They will then have the option to instantly view their results with a single click. This feature will provide valuable insights into participants' performance in detecting normal and abnormal conditions based on the presented images, FIG. 24(a) shows stress, non-stress, and FIG. 24(b) shows murmur, non-murmur.

The FBFT offers certain advantages over the Continuous Wavelet Transform (CWT) and Fast Fourier Transform (FFT) for specific types of signal analysis. Here are some advantages of FBFT compared to CWT and FFT:

Time-Frequency Localization with High Resolution: FBFT provides excellent time-frequency localization, often with higher resolution than CWT and FFT. This means it can capture fine-grained details of how the frequency content of a signal changes over time. This is particularly useful when analyzing signals with rapidly changing or non-stationary spectral characteristics.

Fractal and Self-Similar Signal Analysis: FBFT is well-suited for analyzing signals with fractal or self-similar proper-ties. It can capture and quantify the self-similarity at different scales within the signal, providing insights into the underlying fractal nature of the data. This is not a common feature of CWT and FFT.

Adaptability to Signal Structure: FBFT adapts to the inherent structure of the signal by analyzing it in small overlapping segments. This adaptability allows it to provide meaningful information even for complex signals with varying spectral content.

Understanding Time-Varying Spectra: FBFT is effective for analyzing time-varying spectral content, making it suitable for applications such as analyzing non-stationary signals, environmental data, and financial time series, where the spectral characteristics evolve over time.

The Forward Backward Fourier Transform (FBFT) is a powerful signal analysis technique that excels in revealing intricate details within complex and non-stationary signals. Unlike traditional methods such as the Fast Fourier Transform (FFT) or the Continuous Wavelet Transform (CWT), FBFT provides exceptional time-frequency localization, allowing it to dissect a signal into small, overlapping segments and unveil how its spectral characteristics evolve over time. This high-resolution approach makes FBFT particularly suited for deciphering signals with rapidly changing frequency content or exhibiting fractal and self-similar properties. FBFT serves as a versatile tool for a wide range of applications, from the analysis of financial time series to the investigation of environmental data, enabling a deeper understanding of the underlying dynamics and complexities hidden within signals.

In conclusion, this study has introduced a novel approach and algorithm for enhancing feature extraction techniques of EEG signals, as well as the classification of brain diseases using Convolutional Neural Networks (CNNs) and the interpretability of visual signals in medical image classification. By leveraging deep learning techniques, this disclosure has demonstrated the capability to extract and visualize meaningful features from medical images, enabling medical professionals to gain a deeper understanding of the underlying information. Moreover, the integration of domain-specific knowledge through eye-naked classification has further enhanced the interpretability and diagnostic accuracy of these visual representations.

The combination of these approaches holds great potential to revolutionize medical image classification, leading to more precise diagnoses and improved patient care. However, further investigation is required to fully explore the capabilities of deep learning techniques and eye-naked classification in enhancing the interpretability of visual signals in medical practice.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims

The invention is claimed as follows:

1. A model for brain disease detection based on a forward backward Fourier transform (FBFT) comprising:
a preprocessing function,
a feature extraction function, and
a forward backward Fourier transform (FBFT),
wherein the preprocessing signal receives input EEG signals,
wherein the preprocessing function computes the correlation coefficient for the input EEG signals,
wherein the feature extraction function comprises a Fast Fourier Transformation (FFT), a Power Spectrum (PS), a Continuous Wavelet Transformation (CWT), a Discrete Wavelet Transform (DWT), and a Progressive Fourier Transform (PFT), and
wherein the forward backward Fourier transform (FBFT) uses the equation:
$$X ( f , u ) = min \{ {\int }_{- ∞}^{+ ∞} {e}^{- j 2 π f t} x ( t ) {1}_{\{ t < u \}} dt , {\int }_{- ∞}^{+ ∞} {e}^{- j 2 π f t} x ( t ) {1}_{\{ t > u \} \}} dt \}$$
where X(f,u) represents the result of the operation or transformation for a given frequency f and time u, f represents the signal frequency, u represents the time variable, e is the mathematical constant that represents the base of the natural logarithm, j is an imaginary unit, x(t) is the input signal as a function of time t, 1{t<u} is an indicator function that equals 1 if t belongs to the interval u and is zero otherwise.

2. The system of claim 1, wherein the correlation coefficient between two EEG channels X1 and X2 is calculated by the equation:
$${r}_{X 1 X 2} = \frac{{\sum }_{i = 1}^{n} ( {x}_{1 i} - \overline{{x}_{1}} ) ( {x}_{2 i} - \overline{{x}_{2}} )}{\sqrt{{\sum }_{i = 1}^{n} {( {x}_{1 i} - \overline{{x}_{1}} )}^{2} {\sum }_{i = 1}^{n} {( {x}_{2 i} - \overline{{x}_{2}} )}^{2}}}$$
where n is the total number of samples, x1 and x2 are the mean and x1i, x2i are the samples of the two EEG channels.

3. The system of claim 2, wherein a correlation coefficient is computed for each of the two EEG channels with all other channels resulting in a correlation matrix (CorrMat) given as follows:
$$Corr Mat = [ \begin{matrix}{r}_{X 1 X 1} & {r}_{X 1 X 2} & … & {r}_{X 1 X n} \\ {r}_{X 2 X 1} & {r}_{X 2 X 2} & … & {r}_{X 2 X n} \\ . & & & \\ . & & & \\ {r}_{X nX 1} & {r}_{XnX 2} & … & {r}_{X nX n}\end{matrix} ] ,$$
and
wherein a mean of each column of the CorrMat is then computed to get a single value, which is a mean correlation coefficient for each channel.

4. The system of claim 3, wherein the channels with low mean correlation coefficient values are selected for feature extraction.

5. The system of claim 1, wherein the Fast Fourier Transformation (FFT) is used to calculate Discrete Fourier Transform (DFT) that is used to analyze input EEG signals in the frequency domain, wherein the DFT is calculated at discrete frequencies fn=n, where n=0, 1, 2, . . . , N−1 using the equation:
$$F ( k ) = \frac{1}{N} {\sum }_{k = 0}^{N - 1} x ( n ) {e}^{- j 2 π nk / N} .$$

6. The system of claim 1, wherein Power Spectrum (PS) is the squared absolute value of the Fourier transform with the equation:
$$S ( {f}_{n} ) = \frac{1}{N} {\vert {\sum }_{k = 0}^{N - 1} x ( n ) {e}^{- j 2 π nk / N} \vert}^{2} .$$

7. The system of claim 1, wherein the Continuous Wavelet Transform (CWT) is calculated using the equation:
$${X}_{WT} ( a , b ) = \frac{1}{\sqrt{\vert a \vert}} {\int }_{- ∞}^{∞} x ( t ) {ψ}^{*} ( \frac{t - b}{a} ) dt$$
where * is conjugate in complex number, ψ(t) is the mother wavelet, and a and b are Wavelet coefficients.

8. The system of claim 1, wherein the Discrete Wavelet Transform (DWT) is performed using the Daubechies wavelet with a mother wavelet of db4, wherein it is expressed by:
$${ψ}_{i , k} ( u ) = {2}^{- i / 2} ψ ( {2}^{- i} u - k ) ,$$
and wherein the coefficients are obtained using the equation:
$${W}_{i , k} = W ( {2}^{i} , k {2}^{i} ) = {2}^{- i / 2} {\int }_{- ∞}^{∞} f ( u ) \overline{ψ ( {2}^{- i} u - k )} du .$$

9. The system of claim 1, wherein Progressive Fourier Transform (PFT) is calculated using the equation:
$$F ( f , S ) = {\int }_{- ∞}^{∞} f ( x ) {1}_{x \leq S} {e}^{- j 2 π τ} d τ , S ∈ R .$$

10. A model for brain disease detection based on a forward backward Fourier transform (FBFT) comprising:
a preprocessing function,
a feature extraction function, and
a forward backward Fourier transform (FBFT),
wherein the preprocessing signal receives input EEG signals,
wherein the preprocessing function computes the correlation coefficient for the input EEG signals, and
wherein the forward backward Fourier transform (FBFT) uses the equation:
$$X ( f , u ) = min \{ {\int }_{- ∞}^{+ ∞} {e}^{- j 2 π f t} x ( t ) {1}_{\{ t < u \}} dt , {\int }_{- ∞}^{+ ∞} {e}^{- j 2 π f t} x ( t ) {1}_{\{ t > u \} \}} dt \}$$
where X(f,u) represents the result of the operation or transformation for a given frequency f and time u, f represents the signal frequency, u represents the time variable, e is the mathematical constant that represents the base of the natural logarithm, j is an imaginary unit, x(t) is the input signal as a function of time t, 1{t<u} is an indicator function that equals 1 if t belongs to the interval u and is zero otherwise.

11. The system of claim 10, wherein the correlation coefficient between two EEG channels X1 and X2 is calculated by the equation:
$${r}^{} X 1 X 2 = \frac{{\sum }_{i = 1}^{n} ( {x}_{1 i} - \overline{{x}_{1}} ) ( {x}_{2 i} - \overline{{x}_{2}} )}{\sqrt{{\sum }_{i = 1}^{n} {( {x}_{1 i} - \overline{{x}_{1}} )}^{2} {\sum }_{i = 1}^{n} {( {x}_{2 i} - \overline{{x}_{2}} )}^{2}}}$$
where n is the total number of samples, x1 and x2 are the mean and x1i, x2i are the samples of the two EEG channels.

12. The system of claim 11, wherein a correlation coefficient is computed for each of the two EEG channels with all other channels resulting in a correlation matrix (CorrMat) given as follows:
$$Corr M at = [ \begin{matrix}{r}^{} X 1 X 1 & {r}^{} X 1 X 2 & … & {r}^{} X 1 Xn \\ {r}^{} X 2 X 1 & {r}^{} X 2 X 2 & … & {r}^{} X 2 Xn \\ \cdot & & & \\ \cdot & & & \\ {r}^{} X nX 1 & {r}^{} X nX 2 & … & {r}^{} X nX n\end{matrix} ] ,$$
wherein mean of each column of the CorrMat is then computed to get a single value, which is the mean correlation coefficient for each channel, and wherein the channels with low mean correlation coefficient values are selected for feature extraction.

13. The system of claim 10, wherein the feature extraction function comprises a Fast Fourier Transformation (FFT), a Power Spectrum (PS), a Continuous Wavelet Transformation (CWT), a Discrete Wavelet Transform (DWT), and a Progressive Fourier Transform (PFT).

14. The system of claim 13, wherein the Fast Fourier Transformation (FFT) is used to calculate Discrete Fourier Transform (DFT) that is used to analyze input EEG signals in the frequency domain, wherein the DFT is calculated at discrete frequencies fn=n, where n=0, 1, 2, . . . , N−1 using the equation:
$$F ( k ) = \frac{1}{N} {\sum }_{k = 0}^{N - 1} x ( n ) {e}^{- j 2 π nk / N} ,$$
and
wherein Power Spectrum (PS) is the squared absolute value of the Fourier transform with the equation:
$$S ( {f}_{n} ) = \frac{1}{N} {\vert {\sum }_{k = 0}^{N - 1} x ( n ) {e}^{- j 2 π nk / N} \vert}^{2} .$$

15. The system of claim 14, wherein the Continuous Wavelet Transform (CWT) is calculated using the equation:
$${X}_{WT} ( a , b ) = \frac{1}{\sqrt{\vert a \vert}} {\int }_{- ∞}^{∞} x ( t ) φ * ( \frac{t - b}{a} ) dt$$
where * is conjugate in complex number, ψ(t) is the mother wavelet, and a and b are Wavelet coefficients, and wherein the Discrete Wavelet Transform (DWT) is performed using the Daubechies wavelet with a mother wavelet of db4, wherein it is expressed by:
$${φ}_{i , k} ( u ) = {2}^{- i / 2} φ ( {2}^{- i} u - k ) ,$$
and wherein the coefficients are obtained using the equation:
$${W}_{i , k} = W ( {2}^{i} , k {2}^{i} ) = {2}^{- i / 2} {\int }_{- ∞}^{∞} f ( u ) \overline{φ ( {2}^{- i} u - k )} du .$$

16. The system of claim 14, wherein Progressive Fourier Transform (PFT) is calculated using the equation:
$$F ( f , S ) = {\int }_{- ∞}^{∞} f ( x ) {1}_{x} \leq {s}^{{e}^{- j 2 πτ}} d τ , S ∈ R .$$

17. A method of detecting brain disease using a model for brain disease detection based on a forward backward Fourier transform (FBFT), the method comprising:
receiving input EEG signals,
preprocessing the input EEG signals using a preprocessing function to result in preprocessed signals,
running the preprocessed signals through a feature extraction function to result in extracted signals, and
running the extracted signals through a forward backward Fourier transform (FBFT),
wherein the preprocessing function computes the correlation coefficient for the input EEG signals,
wherein the forward backward Fourier transform (FBFT) uses the equation:
$$X ( f , u ) = min \{ {\int }_{- ∞}^{+ ∞} {e}^{- j 2 π ft} x ( t ) {1}_{\{ t < u \}} dt , {\int }_{- ∞}^{+ ∞} {e}^{- j 2 π ft} 𝒳 ( t ) {1}_{\{ t < u \}} dt \}$$
where X(f,u) represents the result of the operation or transformation for a given frequency f and time u, f represents the signal frequency, u represents the time variable, e is the mathematical constant that represents the base of the natural logarithm, j is an imaginary unit, x(t) is the input signal as a function of time t, 1{t<u} is an indicator function that equals 1 if t belongs to the interval u and is zero otherwise, and
wherein the feature extraction function comprises a Fast Fourier Transformation (FFT), a Power Spectrum (PS), a Continuous Wavelet Transformation (CWT), a Discrete Wavelet Transform (DWT), and a Progressive Fourier Transform (PFT).

18. The method of claim 17, wherein the correlation coefficient between two EEG channels X1 and X2 is calculated by the equation:
$${r}^{} X 1 X 2 = \frac{{\sum }_{i = 1}^{n} ( {x}_{1 i} - \overline{{x}_{1}} ) ( {x}_{2 i} - \overline{{x}_{2}} )}{\sqrt{{\sum }_{i = 1}^{n} {( {x}_{1 i} - \overline{{x}_{1}} )}^{2} {\sum }_{i = 1}^{n} {( {x}_{2 i} - \overline{{x}_{2}} )}^{2}}}$$
where n is the total number of samples, x1 and x2 are the mean and x1i, x2i are the samples of the two EEG channels, wherein a correlation coefficient is computed for each of the two EEG channels with all other channels resulting in a correlation matrix (CorrMat) given as follows:
$$Corr M at = [ \begin{matrix}{r}^{} X 1 X 1 & {r}^{} X 1 X 2 & … & {r}^{} X 1 Xn \\ {r}^{} X 2 X 1 & {r}^{} X 2 X 2 & … & {r}^{} X 2 Xn \\ \cdot & & & \\ \cdot & & & \\ {r}^{} X nX 1 & {r}^{} X nX 2 & … & {r}^{} X nX n\end{matrix} ] ,$$
wherein a mean of each column of the CorrMat is then computed to get a single value, which is a mean correlation coefficient for each channel, and wherein the channels with low mean correlation coefficient values are selected for feature extraction.

19. The system of claim 17, wherein the Fast Fourier Transformation (FFT) is used to calculate Discrete Fourier Transform (DFT) that is used to analyze input EEG signals in the frequency domain, wherein the DFT is calculated at discrete frequencies fn=n, where n=0, 1, 2, . . . , N−1 using the equation:
$$F ( k ) = \frac{1}{N} {\sum }_{k = 0}^{N - 1} x ( n ) {e}^{- j 2 π nk / N} ,$$
wherein Power Spectrum (PS) is the squared absolute value of the Fourier transform with the equation:
$$S ( {f}_{n} ) = \frac{1}{N} {\vert {\sum }_{k = 0}^{N - 1} x ( n ) {e}^{- j 2 π nk / N} \vert}^{2} ,$$
and
wherein Progressive Fourier Transform (PFT) is calculated using the equation:
$$F ( f , S ) = {\int }_{- ∞}^{∞} f ( x ) {1}_{x} \leq {s}^{{e}^{- j 2 πτ}} d τ , S ∈ R .$$

20. The system of claim 14, wherein the Continuous Wavelet Transform (CWT) is calculated using the equation:
$${X}_{WT} ( a , b ) = \frac{1}{\sqrt{\vert a \vert}} {\int }_{- ∞}^{∞} x ( t ) φ * ( \frac{t - b}{a} ) dt$$
where * is conjugate in complex number, ψ(t) is the mother wavelet, and a and b are Wavelet coefficients, and wherein the Discrete Wavelet Transform (DWT) is performed using the Daubechies wavelet with a mother wavelet of db4, wherein it is expressed by:
$${ψ}_{i , k} ( u ) = {2}^{- i / 2} ψ ( {2}^{- i} u - k ) .$$
and wherein the coefficients are obtained using the equation:
$${W}_{i , k} = W ( {2}^{i} , k {2}^{i} ) = {2}^{- i / 2} {\int }_{- ∞}^{∞} f ( u ) \overline{ψ ( {2}^{- i} u - k )} du .$$
